(19) United States
(12) Patent Application Publication
(10) Pub. No.: US 2021/0233671 A1
(43) Pub. Date: Jul. 29, 2021

(54) SYSTEM FOR MANAGING MEDICAL DOCUMENTATION

(71) Applicant: 2659656 Ontario Limited, Ottawa (CA)

(72) Inventor: Matthew James Trevor HERBERT, Ottawa (CA)

(21) Appl. No.: 17/232,705

(22) Filed: Apr. 16, 2021

Related U.S. Application Data
(63) Continuation of application No. PCT/CA2019/051466, filed on Oct. 16, 2019.
(60) Provisional application No. 62/746,235, filed on Oct. 16, 2018.

(51) Int. Cl. G16H 80/00; G16H 20/10; H04L 9/06

(52) U.S. Cl. CPC G16H 80/00; H04L 2209/38; H04L 9/0618; G16H 20/10

(57) ABSTRACT

Systems and methods relating to the management of medical documentation. A main server interfaces with devices used by one or more users, one or more medical practitioners, and one or more medical retailers. The medical practitioner uploads an authorization communication authorizing a user to purchase specific medication from a medical retailer. The authorization communication is stored in a document database and the authorization is communicated to the medication retailer. The user can then purchase/order the medication using the main server. Once ordered/purchased, the medication retailer can then ship/send the medication to the user.

### TECHNICAL FIELD

The present invention relates to medical documentation management. More specifically, the present invention relates to systems and methods for managing medical authorization documentation and online medical records management.

### BACKGROUND

The technological and communications revolution of the early 21st century has had a transformative effect on many industries. With convenience and ease of access being the watchwords of the day and with the Internet almost becoming ubiquitous, consumers can now access most things online. Everything from groceries to clothing to electronics can now be ordered online and be delivered to the consumer in almost record time.

However, even with these changes and the technologies available, retail pharmacy still has not changed. The process is the same as it was decades ago: a patient sees a medical practitioner who issues a prescription, the patient takes the prescription to the pharmacy which then fills the prescription. For prescription refills, the pharmacy may sometimes need to phone or fax the medical practitioner who sends back a refill prescription. As can be imagined, this process is not only tedious but it also quite inefficient.

With the rise of using previously banned substances as medication (e.g. cannabis or cannabis derived medications), the above system is nigh unworkable. Patients have to physically visit their medical practitioners to obtain refill prescriptions for such medications and then have to visit specific pharmacies that are licensed to dispense these medications.

There is therefore a need for systems and methods that allow for a convenient, efficient, and workable system for dispensing medications to patients.

### SUMMARY

The present invention provides systems and methods relating to the management of medical documentation. A main server interfaces with devices used by one or more users, one or more medical practitioners, and one or more medical retailers. The medical practitioner uploads an authorization communication authorizing a user to purchase specific medication from a medical retailer. The authorization communication is stored in a document database and the authorization is communicated to the medication retailer. The user can then purchase/order the medication using the main server. Once ordered/purchased, the medication retailer can then ship/send the medication to the user.

In a first aspect, the present invention provides a system for managing medical documentation, the system comprising:
a main server configured to:
   communicate with medication retailers;
   receive authorization communications from medical practitioners;
   communicate with at least one user to receive orders for medication;
   store said authorization communications in an encrypted format in a database;
wherein
said authorization communications authorizes said at least one user to purchase said medication from at least one of said medication retailers;
said server communicates with said medication retailers to facilitate purchases by said at least one user of said medication from said medication retailers.

In a second aspect, the present invention provides a system for managing medical records for at least one user, the system comprising:
a records management module for communicating with at least one database for storage and retrieval of authorization communications, said authorization communications being communications from medical practitioners that authorizes at least one user to purchase specific medication from medication retailers;
a practitioner communications module for facilitating communications between said system and said medical practitioners to thereby allow said system to receive said authorization communications; and
a retailer communications module for facilitating communications between said medication retailers to thereby allow said system to facilitate purchases for said specific medication for said at least one user.

In a further aspect, the present invention provides a system for verifying identities, the system comprising:
a records management module for communicating with at least one database for storage and retrieval of medical documentation, said medical documentation including authorization communications that are communications from medical practitioners that authorizes at least one user to purchase specific medication from medication retailers;
a practitioner communications module for facilitating communications between said system and said medical practitioners to thereby allow said system to receive said authorization communications; and
a retailer communications module for facilitating communications between said medication retailers to thereby allow said system to facilitate purchases for said specific medication for said at least one user;
   wherein
said at least one database also stores medical records for said at least one user;
identities of said medical practitioners and of said at least one user are continuously verified and updated in said at least one database; and
selected third parties are granted access to query said at least one database for identification verification.

### BRIEF DESCRIPTION OF THE DRAWINGS

The embodiments of the present invention will now be described by reference to the following figures, in which identical reference numerals in different figures indicate identical elements and in which:

FIG. 1 is a schematic block diagram illustrating one aspect of the present invention;

FIG. 2 is a screenshot of a user/patient profile as viewed using one implementation of one aspect of the present invention;

FIG. 3 is a screenshot of a medication search screen as viewed using one implementation of one aspect of the present invention; and

FIG. 4 is a block diagram illustrating the various modules in another aspect of the present invention.

### DETAILED DESCRIPTION

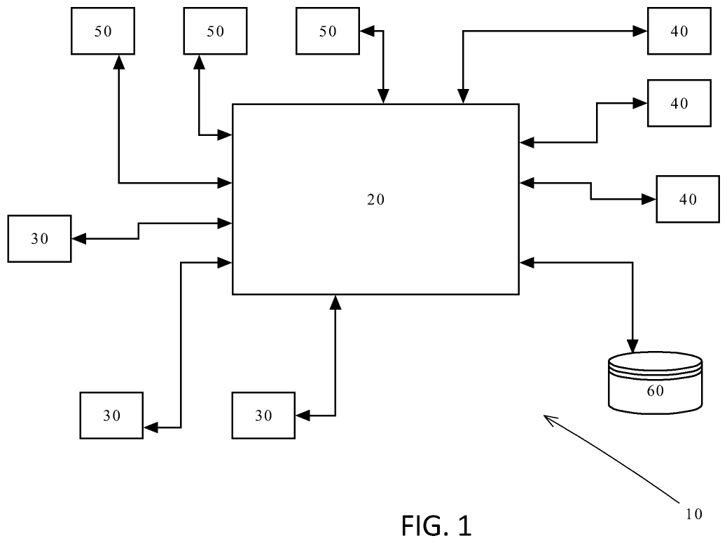

Referring to FIG. 1, a block diagram schematically illustrates a system 10 that operates to provide document management services as well as e-commerce facilitation for medication retailers. As can be seen, the system 10 uses a main server 20 that communicates with medical practitioners and/or health professionals 30 as well as with medication retailers 40. The main server 20 may receive authorization communications, including authorization communications documentation, from the medical practitioners. These authorization communications serve to authorize one or more users 50 to purchase specific medications from the medication retailers. The authorization communications and assorted related documentation are stored/saved by the main server 20 in a database 60.

The system according to one aspect of the present invention operates by managing the documentation and/or the medical records of specific user/patients. In FIG. 1, the medical practitioner sends an authorization communication to the main server with the authorization communication authorizing a specific user to purchase a specific medication. The user who has been authorized to purchase the specific medication can then login to the main server and select one or more medication retailers who sell the specific medication. Once this selected medication retailer has been selected, the main server can forward the authorization communication to the selected medication retailer or, alternatively, the main server can simply notify the selected medication retailer that the main server has the authorization communication that authorizes the specific user to purchase the specific medication. The main server can, in addition to this, notify the medication retailer as to the limits of the authorization communication as necessary. As an example, the main server may notify the selected medication retailer that only a certain amount of the specific medication may be purchased at one time or that only a specific amount may be purchased within a given time span. As well, the main server may notify the medication retailer as to the dosage regimen, bulk amount allowed for purchase, and any other details that the authorization communication may have regarding the specific medication. In addition to this, the main server may forward any other information and/or documentation to the medication retailer that may be authorized or required. The information and/or documentation may, for example, be required to register the user/patient and/or fulfil their orders.

It should be clear that the term “user” includes patients and end users who purchase/receive/order medications from medication retailers and who receive services from the system. This term includes persons and entities who are authorized to act and/or operate on behalf of the end user/patient. This includes caregivers, authorized representatives and authorized relatives of the end user. Thus, even though this document refers to “users”, it should be clear that the person referred to as a “user” may be the end user's representative or authorized intermediary and not necessarily the medication end user himself or herself.

In addition to the above, it should be clear that the term “medication retailer” includes retailers who sell medications to end users and retailers who may also sell medications to other entities such as other medication retailers. This includes medication manufacturers, medication distributers, and other entities in the medication supply chain as well as pharmacies, authorized drug stores, and other entities authorized to retail medication. Such medication retailers would be registered with and vetted/authenticated with the online system of the present invention. It should also be clear that reference to “medication retailer” in this document includes that retailer's employees, agents, and any individuals or entities who may be acting on behalf of that medication retailer.

The term “medical practitioner” includes those who have the authority to prescribe medications and medicaments to the end user of such medications and medicaments. As such, medical doctors and duly appointed registered nurses who have prescription authorization are included under this umbrella term. Such medical practitioners can create and upload prescriptions (e.g. authorization communications) and other documentation about an end user (i.e. the person to whom the medication is administered or who consumes the medication) to the system. Such medical practitioners can, of course, edit the profiles of these end users on the system who are their patients and/or end users who have given their informed consent to the medical practitioner.

For clarity, the term “health professional” includes professionals in the medical and/or health industry who do not have the authority to create prescriptions. Such personnel may include nurses who can dispense but not prescribe medications, caregivers, administrative staff at a hospital, etc., etc. Authorized health professionals using the system are authorized to change/adjust and/or edit the profiles of users on the system and may onboard users to the system. However, it should be clear that these health professionals do not generate authorized communications but may upload such documentation to the system on behalf of the user and/or medical practitioner.

Once the medication retailer has been sent the authorization communication or has been notified of this communication, and has received any other necessary information or documentation, the user can then either login to the medication retailer's website to purchase the medication or, in some implementations, the user can login to the main server of the present invention to purchase the medication. In some circumstances, especially where medical insurance is involved and where an insurance company covers the cost of the specific medication, the medication retailer may simply ship the specific medication to the user. The medication retailer can then invoice the relevant insurance company for the specific medication. Of course, this scenario assumes that there is a pre-existing payment arrangement between the medication retailer and the insurance company for the medication.

In implementations where the user uses the main server to purchase the medication, the main server can be configured to accept payment from the user and to then send out instructions for the selected medication retailer to ship the medication to the user. The main server can then initiate actions to forward/send the user's payment to the medication retailer.

It should also be clear that the main server may operate as a personal record/medical record/medical documentation repository for users. For such an implementation, the user, medical practitioner, or a suitable health professional can send/upload a user's medical records or medical documentation to the database, by way of the main server, for storage. The user, medical practitioner, or other authorized parties such as health professionals, when necessary, can then retrieve these records or documentation from the database, again, by way of the main server.

In other implementations, governmental authorities or other watchdog authorities may also be communicated with by the system and/or given access to the system for regulatory purposes. This may include record checking the medical practitioners, the medication retailers, or even of the main server/system itself. As well, the main server may communicate any other information and/or documentation from the user and/or medical practitioner and/or health professional. The information and/or documentation may, for example, report on adverse reactions to medication and drug interactions. In addition, instead of being provided access to the system for regulatory purposes, some governmental authorities may be provided access to the system to ease the implementation of governmental policies. As an example, providing medications to veterans may be implemented by incorporating the main server as a portal through which these veterans may order medications from licensed/approved medication retailers. It should be clear that, for some implementations, all the medical practitioners, health professionals, and medication retailers registered on the system are checked and/or vetted by a suitable government authority. This ensures that the identity of these entities registered with the system are verified and checked by suitable authorities. In other implementations, instead of government agencies, the vetting and/or checking of identities of the entities registered with the system may be performed by different duly authorized non-governmental authorities such as medical associations, nursing licensing bodies, health and welfare watchdog organizations, and professional associations. This ensures that whoever is registered in the system will have their identities thoroughly checked and verified multiple times both prior to registration with the system as well as while being registered with the system.

Further to the above, the system may also be used in a wider context by being used as a portal for regular patients/users to order/renew prescriptions with regular pharmacies being part of the medication retailers. For such an implementation, the user would merely login to the main server, select a suitable medication retailer (e.g. a pharmacy physically closest to them), and indicate that their medications are running low or have run out. The system would then search for valid authorization communications for that user and that medication (i.e. a valid prescription) in the database and, once it has been found, notify the selected medication retailer about the need for a refill. Alternatively, the user may initially instruct a transfer of the relevant prescription to that medication retailer if required and, once this instruction has been received by the system, the system can initiate the search for the suitable valid authorization communication in its database. The medication retailer would then ship/send the relevant medication to the user with payment to the medication retailer being handled by the medication retailer and/or the user and/or the user's insurance company or benefit provider. As with the above implementations, this assumes that there is a pre-existing arrangement between the medication retailer and the insurance company or benefit provider.

For ease of use, each user of the system is provided with a profile on the system that is accessible to medical practitioners and, where necessary, to other administrative users, health professionals, other medical retailers, or other authorized third parties. Such a profile would detail the user's specifics as well as which medication retailer has been used by the user in the past and which medication retailers have been approved to provide specific medications for the user. This user profile may also include personal information such as the user's contact and address details, health history, current prescriptions, family health history, current and previous medical practitioners and/or health professionals who have had the user under their care, current and previous medication retailers who have sold and/or delivered medications to the user, identifying indicia that uniquely identifies the user, as well as other identity and/or medical related information about the user. As noted above, the data in the user profile may be edited by the user and/or health professionals and/or medical practitioners and/or other third parties who have been explicitly authorized to do so, either by the end user himself/herself or by those who derive their authority from an implicit authorization from the end user. As an example, a nurse assisting a doctor treating the user would be able to amend/edit the user profile as necessary to, for example, detail surgical procedures performed on the user.

It should be clear that all entities registered with the system, including users, medical practitioners, and medication retailers, are provided with suitable profiles in the system. These profiles are stored in the database and provide details regarding the entity. As noted above, the entities are vetted and verified by authorities (both governmental and non-governmental) and the details in the profiles of the entities are thus checked and verified multiple times, both before, during, and after registration with the system. Depending on the implementation of the system, some interactions/transactions with the system (such as registering with the system, transferring users from one medication retailer to another, etc.) may require that the details about the entities involved in the interaction/transaction be verified and/or that the details about the entities be submitted to the system for verification. Note that such verification may be necessary even if these details have been previously checked and vetted. This continuous verification and vetting of the identity of and details about the entities ensures that the database is updated and that those who access the database can be assured that the contents of the database are trustworthy.

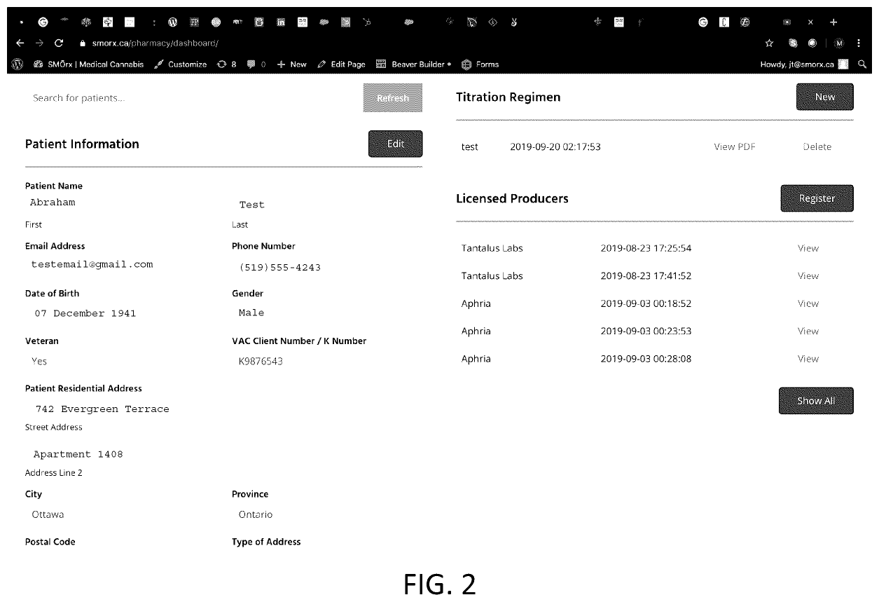

One implementation of the system uses a web-based interface such that system users, whether they be the users or the health professionals or the medical practitioners or representatives of the medication retailers, would only need a web browser to access the main server. Referring to FIG. 2, a sample patient/user profile is provided as experienced/viewed by an individual with administrative access to the system and/or by a medical practitioner/health professional on a web browser. As can be seen, the user's contact information is provided as well as a reference/client number. On the right side of the profile is a list of the licensed producers (i.e., the medication retailers) who have been authorized to sell or who have previously sold medication to the user along with the last date and time that an order has been sent to the medication retailer for this specific user. In addition, the right side of the profile provides the user with access to the prescribed regimen and notification of potential drug interactions for the medication for the specific user/patient. For this example, a single medication is listed. For multiple medications, multiple entries, along with an identification of the medication, would be provided on the user profile. As noted above, medical practitioners are those who are able to prescribe regimen and/or prescribe medication.

For clarity, medication retailers can, in one implementation, access the system via a web-based interface or through a dedicated secure software application in order to send and receive information and documents to other authorized entities on the system.

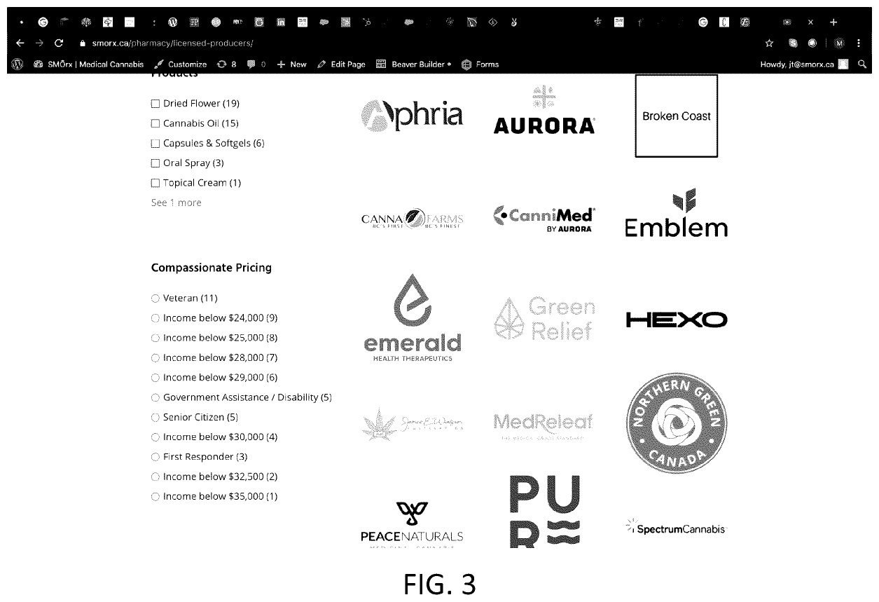

One feature of the present invention is the ability for the user to select a suitable medication retailer for the user's required/desired medication. Assuming there are multiple medication retailers, a user can search the available medication retailers (i.e., the medication retailers available/registered on the system) for service offering and product offerings related to the required/desired medication. While most medications may not allow for a variety of user selectable delivery systems or variants, some medications, such as those derived from cannabis or cannabis itself, may provide for a selection of available products. For some medications, the strength, packaging amount, flavors, price, and other characteristics of the medication product may be selectable by the user. Thus, if a user wants a product that costs a maximum of A dollars per unit, comes in packages of X amount, with a dosage strength of Y, and using a delivery system Z, the user can search for such a product from the available medication retailers. One example of such a search function is illustrated in FIG. 3. As can be seen from FIG. 3, the left side of the interface allows the user to select the criteria for the search. In the screenshot of FIG. 3, the user can select the type of product/form of the product (e.g. dried flower, cannabis oil, capsules, etc.). As well, the user can search for medication retailers that offer compassionate pricing based on specific circumstances (e.g. whether purchaser is a veteran, whether purchaser has income below specific tiers, whether purchaser is a senior citizen, etc., etc.). In addition to being provided with the categories for the search, the search screen already gives an indication of how many of the registered/available medication retailers offer/provide/fall under the available categories. Thus, it can be seen that 19 available medication retailers offer a dried flower formulation for the medication and 11 of the available medication retailers offer compassionate pricing to veterans. The right side of the screen shot shows the user a collection of the available medication retailers.

Another feature of the present invention is that it allows users to also search for a medical professional or other authorized third party (e.g. a health professional or caregiver) to help advise the user or to oversee the user's treatment. Such an advisor, in one implementation, would be authorized to access certain user information and would also be permitted with certain functionality in order to fulfil this role for the user. As noted above, such an advisor may use/interact with the system on the user's behalf.

It should be noted that, while the above mentions a web-based or browser-based implementation for the user interface, different implementations are possible. As an example, different apps or applications may be provided for different classes or types of users to access the system. Thus, a user needing to purchase a refill for his or her medications would need different app than a medical practitioner who wishes to upload/download an authorization communication (e.g. a prescription) for that same user. Similarly, a medication retailer would need a different app to access the system. As well, other authorized third parties would need different apps to access the system. Such an implementation may, of course, be less accessible than a web-based implementation as a web-based implementation would only require that a user have an Internet-connected web browser while an app-based implementation would require that a specific app be downloaded.

Regarding the database noted by the system, multiple types of databases may be used. In one implementation, a blockchain based database was used to ensure an immutable and unchangeable record system. Thus, once an authorization communication has been deposited with the main server, this communication or its representation is used to create a record in a blockchain. This record is then encrypted and propagated through a number of blockchain servers. The actual authorization communication can then be encrypted and stored in a document database with the index to the actual communication being in the blockchain record. Thus, to retrieve a specific authorization communication, the system has to retrieve the blockchain record (from one or more of the blockchain servers), decrypt the record, and use the result to determine the location of the actual authorization communication in the document database.

It should be noted that, in keeping with the blockchain structure of the database, each profile or document may be represented by a block or record in the blockchain and each document (e.g. an authorization communication) can also be represented by a block or record. These blocks or records can be indexed in the blockchain for ease of access to a user's profile. Each block or record can contain an encrypted index that references an encrypted document in the document database. As explained above, all entities registered with the system (i.e., users, medical practitioners, health professionals, medication retailers, etc., etc.) would each have a profile that is stored in the database.

A number of implementation-specific details may be used on the database depending on the desired/required configuration of the system. As an example, the main server may be the root of the blockchain (also known in some circles as a “distributed ledger” system) such that any and all changes to the blockchain (e.g. the addition of a new record) will need to be created/configured by the main server. Once created/configured, a new record or block can be sent from the main server to propagate through the different blockchain servers. In another implementation, each registered/authenticated medical practitioner can be given access to the blockchain such that they can insert/add a new record to the blockchain.

It should be clear that the various aspects of the present invention may be used by any medication dispensing/medication retailing entity as a medication retailer. As well, it is also clear that any type of medication may be used/dispensed/sold using the present invention. However, it has been found that the various aspects of the present invention are quite suitable for the authorized dispensing, sale, and management of medications derived from controlled substances such as cannabis. Thus, the present invention can be used by authorized users to purchase cannabis and cannabis-based or cannabis-derived products from licensed and authorized medication retailers. These medication retailers may be dedicated retailers, producers, or hybrid retailers/producers. For greater clarity, the medication retailer, once a user has purchased the medication, can send the medication to the user by any conventional means such as by post or courier.

One implementation of the present invention also allows the different medication retailers to transact business amongst themselves using the system of the present invention. As an example, the system allows medication retailers to communicate with each other and, when necessary, to select, purchase, and pay for goods/medications from other medication retailers. The system can therefore be used/integrated into the inventory management systems of the various medication retailers to allow for easy resupply, inventory management, and goods transfer between retailers. In one implementation, manufacturers of medications (who also retail to users of the system) can provide medications to other medication retailers who, for their part, simply retail to the system users or to the general public. Thus, in addition to being a portal through which users can purchase medications from medication retailers (who may be manufacturing the medication), the system also allows medication retailers to directly purchase from medication manufacturers. Funds transfers may, depending on the implementation, also be effected using the system.

In another aspect of the present invention, the system allows for transfers of responsibility/authorization between medication retailers. This aspect allows for users to transfer their authorization to purchase medications from one medication retailer to another medication retailer. This transfer may be effected by the user notifying the system that they wish to transfer their authorization to purchase from medication retailer A to medication retailer B. The system then sends one or more confirmatory messages/communications to the user that queries the user whether they have requested such an authorization transfer from A to B and to confirm that the user consents to such a transfer. Upon receipt of such confirmation, the system then queries the medication retailer B (the transferee) as to whether they consent to be the supplier for the medication to the user. For this query, medication retailer B is provided with the necessary details of the user and the medication, including dosage, type of medication, frequency, etc., etc. For some implementations, the transferee medication retailer is provided with the history of the user's purchases from the transferor medication retailer. This ensures that the transferee has complete knowledge of the background of the transfer. In some implementations, the transferee medication retailer may be required to receive identification details about the transferring user from the transferring user himself. This allows for a verification of the transferring user's identity as well as for a verification of other details about the transferring user. In addition, should the transferee medication retailer not have a validation of the medical practitioner who produced the authorization communication that is being transferred, some implementations may require the transferee medication retailer to validate/confirm that medical practitioner's identity. Again, this allows for a continuous validation/checking of the identity and/or credentials of the medical practitioner.

Assuming that the transferee medication retailer consents to such a transfer and assuming that the transferee medication retailer has provided supporting documentation about the consent, the system then sends the consent documentation for the transfer from the client and the transferee medication retailer to the transferor medication retailer (i.e. to medication retailer A). This notifies the transferor medication retailer about the upcoming transfer and that both the transferee and the user have consented to the transfer. The transferor medication retailer then confirms that the transfer can proceed. Of course, the transferor medication retailer may not consent to the transfer because of unpaid invoices, issues with the user, or some other reason. If this occurs, then the system can note the exception and forward the matter to one or more human exception handlers. However, once such confirmation from the transferor medication retailer has been received by the system, the system can then reflect the transfer in its records to specify that, for the specific medication, transferee medication retailer is the supplier for that specific medication. If necessary, the system can also forward the relevant stored authorization communication (e.g. the prescription) from the medical practitioner for this user to the transferee medication retailer. This may ensure that the transferee medication retailer has a copy of the relevant authorization communication that authorizes the specific user to purchase the medication. As well, the system may send any suitable and/or relevant documentation in its database or authorized by the user to the transferee medication retailer as necessary. Of course, this does not mean that all records in the database regarding the specific user is sent to the transferee medication retailer. Rather, the records that would be relevant to and may affect or impact the dispensing and/or purchase of the relevant medication to the specific user would be sent such that the transferee medication retailer would have sufficient documentation for the specific user.

Once the transfer from transferor medication retailer to transferee medication retailer has been effected, the transferee medication retailer can then register the specific user as a client. This may involve the transferee medication retailer contacting the relevant insurance/government agencies, registering the specific user as a client, updating its databases, and confirming/updating the specific user's delivery address/contact information. Where necessary, the system can assist in facilitating contact between the specific user and the transferee medication retailer.

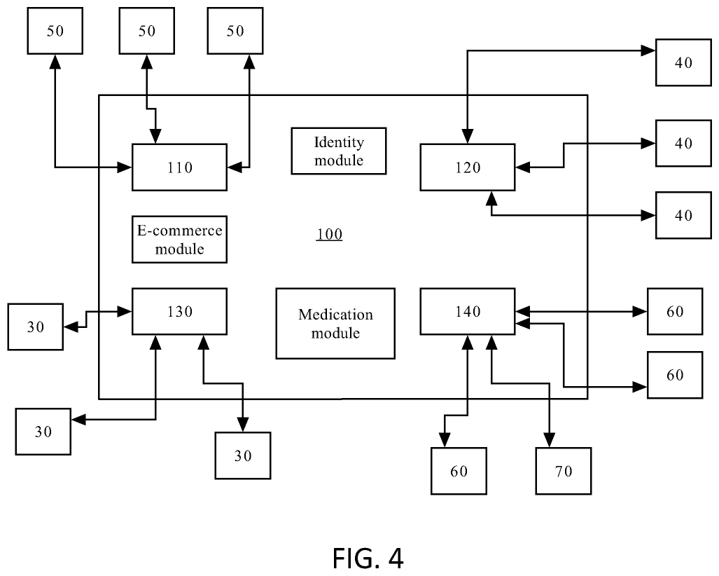

Regarding the main server, this physical/logical construct may be made of multiple servers that operate as a logical single server with multiple functions. Accordingly, multiple modules, with each module performing multiple functions, may be present within the main server construction. In one implementation, the modules present within the main server are those as illustrated in FIG. 4. The presence or absence of these modules within the main server may, of course, depend on the implementation and/or configuration of the main server.

Referring to FIG. 4, it can be seen that the main server 100 includes a user communication module 110, a retailer communications module 120, a practitioner communications module 130, and a records management module 140. These modules operate to communicate and coordinate with the various users of the system as will be explained below. It should be clear that the system allows for communications between the various registered entities of the system. Thus, medication retailers can communicate with medical practitioners, health professionals can communicate with users, and any entity registered with the system can communicate with any other entity similarly registered with the system, as long as the receiving entity has enabled communications with other entities.

As explained above, the user interface for the system may take the form of a web browser-based user experience. When logging in, the person using the system logs in as a user (i.e. a patient), a representative of a medication retailer, or as a medical practitioner (or a representative thereof), or as an authorized health professional. As noted above, the term “user” encompasses a multitude of individuals who derive their authority to use the system directly or indirectly from the actual user. Similarly, representatives of other entities (e.g., medication retailer, medical practitioner, etc.) may use the system on behalf of one of these entities as long as authority to do so is clear. Using a suitable username and password, the system classifies the person logging in and hands the communications over to the relevant communications module. The relevant communications module can then operate to provide the various options available to the person as dictated by that person's role/user category in the system. Thus, as a medication retailer, the person logging in can be provided with options such as managing the medication retailer's account, managing the documentation associated with the medication retailer's account (including authorization communications for specific users/patients, and/or registration instructions and/or transfer instructions), managing user/patients registered as authorized purchasers for the specific medication retailer, managing communications with users and/or other parties authorized to access the system, and managing the available medications listed for the medication retailer on the system. (It should be clear that a medication retailer's products available for order/purchase on the system by users may not constitute all of the medication retailer's products.)

Conversely, as a user/patient, a person logging into the system can be presented with options such as managing the user's account, managing the user's payment options (e.g. by credit card, debit card, etc. if the option to pay is made available by the system configuration), managing which products/medication to purchase from which medication retailers, and managing any standing purchases/orders. As well, the user may be provided with the option as to which medication retailer to use for any/all purchases. The user can also check to determine if an expected authorization communication from a medical practitioner has been received by the system and whether the authorization communication has been forwarded to a suitable medication retailer. Such forwarding would, of course, allow the user to order/purchase the medication from a selected medication retailer. Communications between the system and the user would be handled by the communications module 110. As such, this communications module would receive communications data from a user's device and, where necessary, forward such communications to other modules as necessary.

For the medical practitioner or a health professional, logging into the system would allow the practitioner/professional or his/her representative to determine if their user/patient is registered in the system, determine if suitable authorization communications for a specific user/patient has been logged/stored, and whether the user/patient has been ordering/purchasing the relevant medication. In addition, the medical practitioner can be given the option to register a user/patient with the system, upload an authorization communication, upload medical documentation, manage/edit details in a user's profile, and, generally, manage documentation for one or more of the medical practitioner's patients. For health professionals, these same options, less the options regarding prescriptions or authorization communications, are also available once they have logged into the system. It should be clear that while authorization communications include prescriptions, this does not limit authorization communications to simply prescriptions. Authorization communications include any and all communications that allow or direct a user to purchase or otherwise obtain specific medications that may or may not be restricted by prevailing law. Communications between the system and the device used by the medical practitioner or a health professional would be dealt with by the practitioner communications module 130. Any communications to and from the practitioner/professional would be forwarded to the relevant other modules in the system. Thus, authorization communications, once processed, would be forwarded to the database module for processing and storage. Communications to medication retailers or to users would be forwarded to the relevant communications modules. As well, any communications from other entities registered with the system would be received by the practitioner communications module and would be sent to the practitioner.

For the records management module, the module would deal with the storage, retrieval, and/or encryption/decryption of documentation coming from/going to the database. Should a blockchain based database be used, the records management module would also deal with the propagation of relevant blockchain records in the relevant servers 60 as well as the storage of the actual documentation in the documents database 70. In addition, the records management module would, when receiving a document for storage in a blockchain based database, attend to the creation of a document hash, a storage/database hash (used to locate a document within the document database), creation of a blockchain record, storage of the encrypted document in the document database, and storage and propagation of the blockchain record in the blockchain servers. In addition to a records management module, a medication module may also be present to manage communications about and handle information relating to specific medications and one or more specific users. This module would deal with potential and actual medication interactions, uses, efficacies, formulations, and the like. Such documentation may be reported periodically to specific medical practitioners and/or governmental agencies/authorities as necessary.

For medication retailers, any communications to and from these entities would be handled by the retailer communications module. This includes any communications between different medication retailers. Documentation and/or communications coming from the database or from medical practitioners would be routed through the retailer communications module to ensure that the device used by the medication retailer is properly serviced.

For implementations that allow users to pay for their purchases through the system, an e-commerce module may be present. Such an e-commerce module would be used to receive, process, and attend to payments from users. The module would, preferably, be interfaced with an accounting system, either with the system's accounting system or the accounting system of the relevant medication retailer to whom the payment would be transferred to. Or, in some configurations, the e-commerce module may be interfaced with both accounting systems. In another configuration, once the e-commerce module has received a payment from a user (e.g. by the user entering a credit card number or by the user using a third party service such as PayPal™, the e-commerce module would cause funds, relevant receipts, and records to be produced/forwarded. These funds would be forwarded to the medication retailer that dispenses the medication while the receipts and/or records would be automatically sent to the user and/or to the medication retailer. Such an e-commerce module may also be used to manage/allow communications and funds transfers between any of the entities registered with the system. Thus, when necessary, medical practitioners may transfer funds to health professionals, medication retailers may transfer funds to medical practitioners or health professionals, and, of course, users may transfer funds to medication retailers, medical practitioners, or health professionals.

To ensure that only authorized personnel would be able to use the system, suitable verification measures may be implemented to verify the authenticity of the identities of the various potential users of the system. As an example, the user/patients registering with the system may be required to provide proof of residence, identity, and/or supporting documentation as necessary. These may be scanned and uploaded to the system for automated or manual verification. Additionally, credit card information may be necessary to verify addresses, payment information, and identity for the user. For the medical practitioner, such documentation may also be necessary along with registration numbers, membership information, and other identifying data with relevant/suitable governing bodies (e.g. medical doctor licensing bodies, nurse licensing authorities, etc., etc.). These data points can then be automatically or manually verified before the medical practitioner is allowed access to the various portions of the system. For medication retailers, a more rigorous process may be implemented to ensure that they are legitimate producers/retailers of medications that have been duly approved for sale and/or consumption. Preferably, a government authorized authority can provide documentation/approval for each medication retailer. Absent such documentation/approval from a government authority, the system may not allow such a medication retailer to use the system. It should be clear that the data in the database is continuously updated and verified through governmental and non-governmental authorities. Implementations that constantly require users, medical practitioners, health professionals, and medication retailers to continuously and repeatedly verify (whether by themselves or through other agencies) their identities, credentials, and contact information are preferred. Such implementations would ensure that the contents in the database are trustworthy for not just those who are registered in the system but also those who may use the system but are not registered.

The continuous verification and authentication of the various entities registered with the system ensures that the authenticity of the identities of these entities is trustworthy. As such, third parties may use the system to authenticate the identity of individuals who are registered with the system. An extra identity module may be installed in the system to handle such queries such that the system can operate as a validation authority for validating the identity of an individual or an entity. A third party, who may gain limited access to the system through a subscription service, can then login and verify if an entity's identity is legitimate or authentic. Thus, such a third party can query a company's identity and contact information, an individual's identity parameters (e.g. name, address, occupation, contact information, etc.), as well as other parameters relating to an individual's identity, location, or history. Of course, prior to such access, each entity's consent to the existence of such access is required. When registering for the system, an entity (e.g. a user, a medical practitioner, health professional, or a medication retailer) would need to consent to their details being available for searching and queries by third parties. Of course, the amount of detail that would be available to such searching and queries would be determined by the entity whose consent is being sought. This allows for situations where one individual may consent to their medical records being open to general public scrutiny while another individual may only consent to having their name and address being available for queries. The level of queries that the individual consents to would determine how much of their information in the database is open to searches/queries from third parties.

The above system of identity verification allows for third parties to subscribe to an identity verification service such that a third party can query the system and, in return, be provided with details regarding the subject of the query. The continuous verification and confirmation of the details regarding each entity's identity (as outlined above) ensures that the data in the database is trustworthy and that the results of identity queries are similarly trustworthy for third parties.

The large amount of data stored within the database regarding user information, medical information, medication information, medication results, dosage information, etc., etc. also allows for such data to be mined and used for better recommendations, prescriptions, and research. An AI/machine learning module may be used with the present invention to allow the use of the contents of the database as datasets or as part of datasets used to train AI/machine learning devices/systems. Treatment personalization, treatment customization, prescription management, prescription recommendation, optimized product purchasing, optimized product distribution, product efficacy analysis, enterprise operational analysis (for medication retailers), customer/user servicing efficiency analysis, and prescription management/optimization are just some of the fields that can be serviced by such an AI/machine learning devices/systems.

It should be clear that the term “server” encompasses both a physical and a logical server. While some implementations of this aspect of the invention may use a single physical server as the main server, other implementations may use a logical server that is a conglomeration or an aggregation of multiple physical servers to result in a single logical server that accomplishes the various functions of the system. In addition, while the above discussion makes note of a database, the database may be a single physical database or a multitude of databases that have been aggregated or conglomerated into a single logical database.

It should be clear that the various aspects of the present invention may be implemented as software modules in an overall software system. As such, the present invention may thus take the form of computer executable instructions that, when executed, implements various software modules with predefined functions.

The embodiments of the invention may be executed by a computer processor or similar device programmed in the manner of method steps, or may be executed by an electronic system which is provided with means for executing these steps. Similarly, an electronic memory means such as computer diskettes, CD-ROMs, Random Access Memory (RAM), Read Only Memory (ROM) or similar computer software storage media known in the art, may be programmed to execute such method steps. As well, electronic signals representing these method steps may also be transmitted via a communication network.

Embodiments of the invention may be implemented in any conventional computer programming language. For example, preferred embodiments may be implemented in a procedural programming language (e.g., “C” or “Go”) or an object-oriented language (e.g., “C++”, “java”, “PHP”, “PYTHON” or “C#”). Alternative embodiments of the invention may be implemented as pre-programmed hardware elements, other related components, or as a combination of hardware and software components.

Embodiments can be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink-wrapped software), preloaded with a computer system (e.g., on system ROM or fixed disk), or distributed from a server over a network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

A person understanding this invention may now conceive of alternative structures and embodiments or variations of the above all of which are intended to fall within the scope of the invention as defined in the claims that follow.

1-36. (canceled)

37. A system for managing medical documentation, the system comprising a main server configured to:
communicate with one or more medication retailers;
communicate with at least one user to receive one or more orders for one or more medications;
receive an authorization communication from medical practitioners for at least one user;
store said authorization communication, as a stored authorization communication, in an encrypted format in a database;
wherein a processor on said main server processes said orders, by said at least one user, of said one or more medications from said one or more medication retailers as authorized by said stored authorization communication.

38. The system according to claim 37, wherein said medication is cannabis related medication.

39. The system according to claim 37, wherein said authorization communications are stored in a blockchain-based storage subsystem.

40. The system according to claim 37, wherein said medication is purchased by said at least one user from a medication retailer selected by said at least one user.

41. The system according to claim 40, wherein said server communicates with a selected medication retailer selected by said at least one user to confirm that a suitable authorization communication from one of said medical practitioners has been received for said medication being purchased by said at least one user from said selected medication retailer.

42. The system according to claim 37, wherein said main server is further configured to manage medical records for said at least one user.

43. The system according to claim 37, wherein one of said one or more medication retailers is a pharmacy.

44. The system according to claim 38, wherein one of said one or more medication retailers is a producer of cannabis related medication.

45. The system according to claim 37, wherein said main server is further configured to communicate with at least one medical insurer.

46. The system according to claim 37, wherein said main server comprises:
a records management module for communicating with at least one database for storage and retrieval of said stored authorization communication;
a practitioner communications module for facilitating communications with said medical practitioners;
a retailer communications module for facilitating communications with said one or more medication retailers; and
a user communications module for communicating with said at least one user.

47. The system according to claim 46, further comprising an e-commerce module for facilitating said purchases by said at least one user from said one or more medication retailers.

48. The system according to claim 37, wherein stored authorization are transferred between different medication retailers with at least one verification selected from the group consisting of: a user's identity, a medical practitioner's identity, a medical practitioner's license, an identity of a transferee medication retailer, a consent of said transferee medication retailer to receive said authorization for said medications, and a consent of a user to transfer authorization for said medications from one medication retailer to a different specific medication retailer.

49. The system according to claim 37, wherein said system is used further for identity verification by at least one third party.

50. The system according to claim 49, wherein said system is used to verify an identity of an individual who is at least one of: a user, a medical practitioner, a health professional, and a medication retailer.

51. The system according to claim 49, wherein said at least one third party subscribes to a subscription service that provides identity verification using data in said database.

52. A method for managing medical documentation, the method comprising:
communicating, via a main server, with one or more medication retailers;
receiving, via said main server, one or more orders for one or more medications for at least one user;
receiving, at said main server, an authorization communication from a medical practitioner for a medication for said at least one user;
storing, by said main server, said authorization communication, as stored authorization communication, in an encrypted format in a database;
processing, by said main server, orders of said medications, made by said at least one user, from said one or more medication retailers as authorized by said stored authorization communication.

53. The method of claim 52, wherein said one or more medications are cannabis related medications.

54. The method of claim 52, wherein said authorization communication is stored in a blockchain-based storage subsystem.

55. The method according to claim 52, wherein said server communicates with said one or more medication retailers, selected by said at least one user, to confirm that a suitable authorization communication from one of said medical practitioners has been received for said one or more medications being purchased by said at least one users from one of said one or more said medication retailers.

56. The method of claim 52, wherein one of said one or more medication retailers is a pharmacy.
